(19) United States
(12) Patent Application Publication
(10) Pub. No.: US 2006/0210966 A1
(43) Pub. Date: Sep. 21, 2006

(54) Kits and methods for detecting bovine ephemeral fever virus

(75) Inventors: George Chin-Sheng Chou, Tainan Hsien (TW); Shu-Chen Chen, Tainan Hsien (TW); Shu-Wen Hsiao, Tainan Hsien (TW)

Correspondence Address: Dougherty & Troxell, ONE SKYLINE PLACE, 5205 LEESBURG PIKE, SUITE 1404, FALLS CHURCH, VA, 22041, US

(73) Assignee: AsiaGEN Corporation

(21) Appl. No.: 11/082,707

(22) Filed: Mar. 18, 2005

(51) Int. Cl. C12Q 1/70; C12Q 1/68; C12P 19/34

(52) U.S. Cl. 435/5; 435/6; 435/91.2

(57) ABSTRACT

The present invention relates to a kit and method for detecting BEFV of suspected patient. The present invention also relates to primers and probe used to detect BEFV.

### FIELD OF THE INVENTION

The present invention relates to a kit and a method for detecting bovine ephemeral fever virus (BEFV) in a sample by the use of a nested polymerase chain reaction. The present invention also relates to primers and probe for detecting the presence of BEFV.

### BACKGROUND OF THE INVENTION

Bovine ephemeral fever virus (BEFV) is an arthropod-borne rhabdovirus which causes an acute febrile infection in cattle and water buffalo. BEFV virions are bullet-shaped and contain 5 structural proteins: L (Mr=180 kDa); G (Mr=81 kDa); N (Mr=52 kDa); M1 (Mr=43 kDa); and M2 (Mr=29 kDa) (Walker et al., (1991) J. Gen. Virol. 72, 67-74) and one non-structural protein GNS. As for rabies virus and vesicular stomatitis virus, the BEFV membrane glycoprotein (G) can be removed from virions by treatment with non-ionic detergent.

The G protein presents type-specific and neutralizing antigenic sites. Six neutralization sites have been identified by competitive binding of G protein monoclonal antibodies (Cybinski et awl., (1990) J. Gen. Virol. 71, 2065-2072). The virion G protein also protects cattle from experimental infection with virulent BEFV as described in Australian Patent No. 636907.

Bovine ephemeral fever (BEF) is a noncontagious epizootic arthropod-borne viral disease of cattle and water buffaloes characterized by sudden onset of fever, depression, stiffness, and lameness. The disease occurs in most tropical and sub-tropical regions of Africa, Asia, the Middle-East and Australia, where seasonal epidemics can have significant economic consequences.

BEFV infection is part of a cycle whereby infected cattle may be bitten by insects such as mosquitoes and sandflies which may then transmit the infection to healthy animals. There is a sudden onset of fever—as high as 41° C. compared with the normal temperature of about 38° C. The temperature returns to normal within 36 hours. The first sign in milking cows is a sudden and severe drop in milk production. Cows in advanced pregnancy may abort. Animals stop eating and drinking and become depressed. They usually drool saliva, develop a stringy nasal discharge, and may have watery eyes.

Affected animals may shiver and often become very stiff with a shifting lameness, and are reluctant to move. The joints may appear swollen and sometimes there is swelling around the jaw. Some animals—particularly the heavier ones—just lie down and refuse to move.

By day three the affected animal is usually standing again and will begin to eat. However, lameness and weakness may last for another two or three days. In the vast majority of cases the disease runs a short course, followed by rapid and complete recovery. However, the disease can vary in severity. Some animals may show only slight symptoms for about 24 hours, while a small number may stay down for many weeks. The disease is usually milder in calves under 12 months of age.

Milk production usually drops by at least 50% in sick cows. In dairy herds it is the highest producing animals that are usually the most severely affected. Yield should return nearly to normal after about three weeks, but cows affected late in lactation often dry off. Mastitis sometimes develops, with a marked rise in the somatic cell count. Although most of the herd can be affected, deaths from ephemeral fever are uncommon and rarely involve more than 1% of the herd. Death is usually the result of misadventure or being down for a long period.

Clearly, expedient diagnosis is important in controlling the spread of BEF. Traditionally a sample of blood should be taken during the period of fever and a second 1-2 weeks later. Part of the first sample of blood is allowed to clot, and another portion is mixed with anticoagulant. From the uncoagulated blood, a smear is made on a glass slide and allowed to dry in air. The balance is used for virus isolation (wren, M. F., et al. 1992. Vet. Microbiol., 30:297-307). When blood taken during illness is allowed to clot, it usually fails to contract on standing, even over several days. It may be streaked with fibrin. Samples should be taken from animals in various stages of the disease to facilitate a rapid laboratory confirmation.

The most efficient means of proving the identity of the disease is the transmission to susceptible cattle by the intravenous injection of uncoagulated whole blood. These cattle are closely observed for the development of fever and the characteristic signs. Virus isolation can be attempted (from the leukocyte fraction of the blood) in tissue cultures but is not very efficient (Uren, M. F., et al. 1992. Vet. Microbiol., 30:297-307).

A differential leukocyte count on the blood smear provides the most rapid supporting evidence for the field diagnosis. A high percentage of neutrophils with many immature forms are not pathognomonic of ephemeral fever, but if not present the field diagnosis is likely to be wrong. Eosinopenia also occurs.

Testing of antibody (virus-serum neutralization test) is the most generally available laboratory test. However, false positives do occur. In addition, collecting samples for antibody test requires a longer time, therefore not suitable for early stage screening and quarantine.

There are several immunological methods established for the detection of specific antibodies to BEFV (Zakrzewiski, H. et al., 1992, A blocking ELISA for the detection of specific antibodies to bovine ephemeral fever virus. J. Immunol. Methods 151, 289-297; Hsieh, Y. C. et al., 2005. Bovine ephemeral fever virus infection in Taiwan (2001-2002). J. Vet. Med. Sci.). To date, no sensitive, reliable, and quantitative techniques for BEFV were established.

In recent years, nucleic acid detection has become a standard technique for monitoring virus infection. Few copies of viral DNA could be detected in suspected sample before the antibody has rise to significant level. Conventional RT-PCR and Real-time PCR are techniques commonly used in laboratories these days. However, the sensitivity of conventional RT-PCR and real-time RT-PCR are generally not good.

Given the above, current available assay could not quickly and completely detect BEFV. A high-speed assay with high specificity and sensitivity to detect BEFV from available samples is eagerly needed on the market.

### SUMMARY OF THE INVENTION

The present invention provides a kit for detecting the presence or absence of bovine ephemeral fever virus (BEFV) in a sample using a nested polymerase chain reaction, comprising
(i) an outer pair of oligonucleotide primers selected from the group consisting of
(a) SEQ ID NOS: 1 and 2,
(b) SEQ ID NOS: 1 and 3,
(c) SEQ ID NOS: 4 and 2,
(d) SEQ ID NOS: 4 and 3, and
(ii) an inner pair of oligonucleotide primers SEQ ID NOS: 5 and 6.

The present invention further provides a method for detecting the presence or absence of BEFV in a sample using a nested polymerase chain reaction, comprising
(i) adding into one tube with RNA from the sample, reverse transcriptase, buffer, dNTP, Taq polymerase, an outer pair of oligonucleotide primers selected from the group consisting of
   (a) SEQ ID NOS: 1 and 2,
   (b) SEQ ID NOS: 1 and 3,
   (c) SEQ ID NOS: 4 and 2, and
   (d) SEQ ID NOS: 4 and 3,
(ii) performing the first-stage polymerase chain reaction of the PCR products from the outer primers;
(iii) adding into one tube with template from (ii), buffer, dNTP, Taq polymerase, and an inner pair of oligonucleotide primers SEQ ID NOS: 5 and 6;
(iv) performing the second-stage polymerase chain reaction of the PCR products from for the inner primers; and
(v) identifying BEFV by the probe having SEQ ID NO: 7.

The present invention also provides novel nucleotide sequences for detecting the presence or absence of BEFV.

### BRIEF DESCRIPTION OF THE DRAWINGS

FIG. 1 is agarose gel electrophoresis of PCR-amplified cDNA fragment (479 bp) using BEFV genomic RNA extracted by using two different kits. Lane M denotes 100 DNA ladder molecular weight marker; lane NC denotes negative control; lane 1 denotes 5-fold dilution of RNA; lane 2 denotes 10-fold dilution of RNA; lane 3 denotes 1,000-fold dilution of RNA; and lane 4 denotes 10,000-fold dilution of RNA.

FIG. 2 is agarose gel electrophoresis of PCR-amplified cDNA fragments using four different sets of primers. Lane M denotes 100 DNA ladder molecular weight marker; lane NC denotes negative control; lane 1 denotes G2F/G2R1; lane 2 denotes G2F/G2R2; lane 3 denotes G2F2/G2R1; and lane 4 denotes G2F2/G2R2.

FIG. 3 is agarose gel electrophoresis of first and second PCR-amplified cDNA fragments using different annealing temperatures. Lane M denotes 100 DNA ladder molecular weight marker; lane NC denotes negative control; lane 1 denotes 40° C.; lane 2 denotes 45° C.; lane 3: 50° C.; lane 4 denotes 55° C.; lane 5 denotes 60° C.; and lane 6 denotes 65° C.

FIG. 4 is comparison of optimal hybridization buffers (I and II) for the assay of the invention. The Y-axis indicates relative light units (RLU) for tested samples. The values shown are the means of three independent experiments. NC denotes negative control; G2 represents positive control.

FIG. 5 is comparison of optimal temperatures for the assay of the invention. The Y-axis indicates relative light units (RLU) for tested samples. The values shown are the means of three independent experiments. NC denotes negative control; Test represents temperature (50° C. and 55° C.).

FIG. 6 is the sensitivity of the assay of the invention. The Y-axis indicates relative light units (RLU). The X-axis indicates the serial diluted standard plasmid (copy number). The values shown are the means of three independent experiments. NC represents negative control without DNA. PC represents positive control.

FIG. 7 is the specificity of the Assay of the present invention. Five BEFV strains, including 9316, 90126, 9333, 9310, and 9309, were examined. The Y-axis indicates relative light units (RLU). The X-axis indicates the 10-fold dilutions of BEFV-infected cells. The values shown are the means of three independent experiments. NC represents negative control without BEFV. PC represents positive control.

FIG. 8 is the detection of 34 clinical samples suspected to have BEFV infections in cattle by using the Assay of the present invention. The Y-axis indicates the sample-to-negative control ratios for each tested sample. The X-axis indicates the sample no. and a positive control. The values shown are the means of three independent experiments. P represents positive control (BEFV 2004-1/TN/TW).

### DETAILED DESCRIPTION OF THE INVENTION

The assay of the present invention for rapid and sensitive detection of BEFV was developed. This technique uses a nested PCR and magnetic beads-based probing technique, which can improve the sensitivity detection of BEFV. This assay of the present invention showed more sensitive than the conventional RT-PCR and real-time RT-PCR assays for the detection of bovine ephemeral fever viruses from cattle suspected to have BEFV infections.

Previous report suggested that the field isolates in Taiwan exhibited amino acid substitutions in the neutralization epitopes on the surface glycoprotein (Hsieh et al., 2005). The main difficulty in developing a molecular assay for BEFV is the design and construction of the primers because of the genetic variation (Hsieh et al., 2005). Using multiple sequence alignments of BEFV isolates and recently identified variants, highly conserved primers and probes were selected from the G protein-encoding gene of BEFV. Sequence analysis of primers and probe showed no significant homology with any other pathogens. The primers used in the assay of the present invention gave specific and sensitive detection of BEFV as no amplification occurred in PCR reactions using DEPC-treated water or unrelated viruses as negative controls. By applying nested PCR technique, the assay of the present invention is able to detect as little as 1 copy of BEFV plasmid DNA.

Target Gene

The present invention is related to nucleotide sequences comprising highly specific oligonucleotide primers that are synthesized from and hybridize to specific portions of a BEFV G2, G3b and G3c gene having the nucleotide sequence set forth in SEQ ID NOS. 1, 2, 3 and 4.

The nucleotide sequences for preferred outer pair of oligonucleotide primers of the present invention are set forth in SEQ ID NOS. 1 and 2.

In addition, the present invention also provides an inner pair of oligonucleotide primers for nested PCR reaction are set forth in SEQ ID NOS: 5 and 6.

The present invention further provides a novel probe having SEQ ID NO. 7 for identifying the nested PCR products.

Therefore, the present invention provides nucleotide sequence S for detecting the presence or absence of bovine ephemeral fever virus (BEFV) is selected from the group consisting of

| | |
|---|---|
| GACATGGATACCAGAAGTGAGAGTC | (SEQ ID NO: 1) |
| GTAGAGTTCAAAGGATTG | (SEQ ID NO: 2) |
| GGAGAACATCAACAAGAG | (SEQ ID NO: 3) |
| CTCCTTGTACTATTAATGAC | (SEQ ID NO: 4) |
| GGCAGAATTAGAACATGAACG | (SEQ ID NO: 5) |
| GAGCAAACAAGTTGGCAAG | (SEQ ID NO: 6) |
| CCACAAGACCAGGAAGAGATTA. | (SEQ ID NO: 7) |

These single-stranded primers are comprised of nucleotide sequences including naturally occurring nucleotides and any variants thereof. By “naturally occurring nucleotides” is intended adenosine triphosphate, guanosine triphosphate, cytosine triphosphate, thymidine triphosphate, uridine triphosphate, and inosine triphosphate. By “any variants thereof” is intended any nucleotides comprising modified bases of the form N6-(6-aminohexyl) (as in N6-(6-aminohexyl) dATP or N6-(6-aminohexyl) ATP), or comprising bases modified as 5′-thiol, 5′-phospho, 5′-methyl, 5′-biotinylated, 5′-amino, or 5′-fluoro (as in 5′-fluoro-deoxyadenosine).

These primers are designed for desirable characteristics, including inability to form hairpin loops. Additionally, when any two of these primers are used as a primer pair for a polymerase chain reaction method according to the present invention, they do not hybridize to each other. All of these characteristics enable a highly sensitive, highly specific nested polymerase chain reaction approach for detection of the BEFV in potentially infected samples.

Primers for Nested PCR

In an embodiment of the present invention, these primers are used in a nested polymerase chain reaction (PCR) method to detect the presence of the BEFV G2, G3b and G3c genes in a purified sample nucleic acid mixture, the nucleotide sequences of which have been extracted from a potentially infected sample. By “nested” PCR method is intended a two-staged polymerase chain reaction process. In a first-stage polymerase chain reaction, a pair of “outer” oligonucleotide primers, consisting of an upper and a lower primer that flank a particular first “target” nucleotide sequence in the 5′ and 3′ position, respectively, are used to amplify that first sequence. In a second-stage polymerase chain reaction, a second set of “inner” or “nested” oligonucleotide primers, also consisting of an upper and a lower primer, is used to amplify a smaller second “target” nucleotide sequence that is contained within the first target nucleotide sequence.

The upper and lower inner primers flank the second target nucleotide sequence in the 5′ and 3′ positions, respectively. By “flanking primers” is intended primers that are complementary to segments on the 3′-end portions of the double-stranded target nucleotide sequence that is amplified during the PCR process. By “target” nucleotide sequence is intended a nucleotide sequence comprising a predetermined portion of the BEFV G2, G3b and G3c genes set forth in SEQ ID NOS: 1, 2, 3 and 4. The base-pair size of these target nucleotide sequences and their particular position within the BEFV G2, G3b and G3c genes are determined by the pair of outer primers and pair of inner primers used in the first- and second-stage polymerase chain reactions, respectively.

PCR and Nested PCR References

Nested PCR methods are available in the art. See generally U.S. Pat. No. 4,683,202, U.S. Pat. No. 5,487,969 (herpes B virus); U.S. Pat. No. 5,545,523 (bovine herpes virus-1).

Procedures of One Tube Nested PCR—RT-PCR and PCR Amplification

The nested PCR method of the present invention comprises the following steps. In a first-stage RT-PCR, specific outer oligonucleotide primers are added to the sample nucleic acid mixture, and the resulting mixture is subjected to an initial denaturation step to obtain single-stranded DNA templates. Following denaturation, the mixture is subjected to an initial annealing step, where the outer primers hybridize to opposite strands of the first targeted nucleotide sequence. The temperature is then raised to allow for extension or replication of the specific segment of DNA across the region between the two primers by a thermostable DNA polymerase. The reaction is then thermocycled to allow for repeated denaturation, annealing, and extension, so that at each cycle, the amount of DNA representing the targeted nucleotide sequence between the two outer primers is doubled and the BEFV gene such as G2, G3b and G3c are amplified.

The first nucleotide sequence within the BEFV gene such as G2, G3b and G3c, which is targeted for amplification in the first-stage polymerase chain reaction, is flanked by an upper primer in the 5′ upstream position and a lower primer in the 3′ downstream position. The first targeted nucleotide sequence, and hence the amplification product of the first-stage polymerase chain reaction, has a predicted base-pair length, which is determined by the base-pair distance between the 5′ upstream and 3′ downstream hybridization positions of the upper and lower primers, respectively, of the outer primer pair. The upper and lower primers of the outer primer pair are derived from the BEFV gene such as G2, G3b and G3c.

Procedures of One Tube Nested PCR—Second Stage

In this second-stage reaction, the products of the first-stage reaction are combined with specific inner or nested primers. These inner primers are derived from nucleotide sequences within the first targeted nucleotide sequence and flank a second, smaller targeted nucleotide sequence contained within the first targeted nucleotide sequence. This mixture is subjected to initial denaturation, annealing, and extension steps, followed by thermocycling as before to allow for repeated denaturation, annealing, and extension or replication of the second targeted nucleotide sequence. This second targeted nucleotide sequence is flanked by an upper primer in the 5′ upstream position and a lower primer in the 3′ downstream position. The second targeted nucleotide sequence, and hence the amplification product of the second-stage PCR, also has a predicted base-pair length, which is determined by the base-pair distance between the 5′ upstream and 3′ downstream hybridization positions of the upper and lower primers, respectively, of the inner primer pair. The upper and lower primers of the inner primer pair are derived from within the BEFV gene such as G2, G3b and G3c.

Analyze Product of Nested PCR

The amplification products of the first- and second-stage polymerase chain reaction may be analyzed to identify the presence or absence of the first and second targeted nucleotide sequences comprising specific region of the BEFV gene. Identification of the amplification products may be accomplished by any one of several methods known in the art to detect amplified nucleotide sequences. These methods include, but are not limited to, determination of size, restriction enzyme digestion pattern, subsequent cloning of amplification products, Southern blot hybridization, with an oligonucleotide probe internal to the nucleotide sequence being amplified, or DNA sequencing.

Analyze Product of Nested PCR—Electrophoresis

The size of the product or products may be determined by electrophoresis through a gel, preferably an agarose gel, simultaneously with molecular size standards of known base-pair length. The gel may be stained with ethidium bromide, which intercalates between base pairs and enables the visualization of DNA upon illumination with ultraviolet light. In this manner, amplification products from the first- or second-stage PCR having equidistant migration with molecular size standards of approximately the base-pair length of the predicted first or second targeted nucleotide sequence, respectively, would verify presence of the BEFV within the original tissue sample.

Accordingly, the present invention provides a method for detecting the presence or absence of BEFV in a sample using a nested polymerase chain reaction, comprising
(i) adding into one tube with RNA from the sample, reverse transcriptase, buffer, dNTP, Taq polymerase, an outer pair of oligonucleotide primers selected from the group consisting of
   (a) SEQ ID NOS: 1 and 2,
   (b) SEQ ID NOS: 1 and 3,
   (c) SEQ ID NOS: 4 and 2, and
   (d) SEQ ID NOS: 4 and 3,
(ii) performing the first-stage polymerase chain reaction of the PCR products from the outer primers;
(iii) adding into one tube with template from (ii), buffer, dNTP, Taq polymerase, and an inner pair of oligonucleotide primers SEQ ID NOS: 5 and 6;
(iv) performing the second-stage polymerase chain reaction of the PCR products from for the inner primers; and
(v) identifying BEFV by the probe having SEQ ID NO: 7.

In the method of the invention, the probe can be directly detected to BEFV. In the preferred embodiment of the invention, the probe is coupled to magnetic beads. The magnetic beads are covalently coupled to amine-containing oligonucleotides through their surface carboxylate groups. The small size allows the magnetic beads to remain in suspension for several hours, which is more sufficient for assay setup and analysis, and also provides near-fluid-phase reaction kinetics.

To increase sensitivity and specificity of the assay of the present invention, the assay was optimized by using various primer sets and annealing temperatures, as well as hybridization conditions. The rapid kinetics of magnetic beads-based assays allow shorter incubation times than conventional solid-phase assays. The hybridization between oligonucleotide and capture probe-coupled magnetic beads was faster. Incubation as brief as 20 min was efficient in the assay. Therefore, the hybridization time required for assay of the present invention was much shorter than that required for other technologies. The overall time for processing this assay is less than 7 h. The reduced assay time also reduces labor costs for performing the diagnosis of BEFV infections. The speed of the assay, the short hybridization time, and the simple procedures of the assay make it superior to any other technologies.

In general, the hybridization reaction carried out at 30-60° C., under 1-10× standard saline citrate (SSC) buffer and 0.1-2% sodium dodecyl sulphate (SDS). In the preferred embodiment, the hybridization reaction is carried out at 50° C., under 5× standard saline citrate (SSC) buffer and 0.5% sodium dodecyl sulphate (SDS).

A luminometer is used to detect and measure luminescence that normally comes from chemical or biological reactions.

The method of the present invention could be applied to detect the presence of one copy of BEFV in a sample. Especially, the present invention is suitable to detect the early stage of BEFV infections in a sample.

Analyze Product of Nested PCR—Labeled Primers

In another embodiment, labeled primer pairs in the first or/and second stage of amplification could be used in the nested PCR amplification to detect the amplification product directly. Commonly employed labels include, but are not limited to, fluorescent molecules, radioactive molecules, chromogenic substrates, biotin, acridinium ester and acridinium-9-carboxamide. The methods for biotinylating nucleic acids are well known in the art, as are methods for introducing fluorescent molecules and radioactive molecules into oligonucleotides and nucleotides. In the preferred embodiment of the invention, the inner pair of oligonucleotide primers is labeled by biotin.

When biotin is employed, it is detected by avidin, streptavidin or the like, which is conjugated to a detectable marker, such as an enzyme (e.g., horseradish peroxidase). Enzyme conjugates are commercially available from, for example, Vector Laboratories (Burlingame, Calif.). Steptavidin binds with high affinity to biotin, unbound streptavidin is washed away, and the presence of horseradish peroxidase enzyme is then detected using a luminescence-emission substrate in the presence of peroxide and appropriate buffers. The product may be detected using a Berthold Luminometer (Pforzheim, Germany).

Detection methods are well known for fluorescent, radioactive, chemiluminescent, chromogenic labels, as well as other commonly used labels. Briefly, chemiluminescence can be identified and quantitated most directly by their emission wavelengths and intensity.

Analyze Product of Nested PCR—Southern Blot

In still another embodiment, a probe may be used to detect the amplification product of the nested PCR. As used herein, a “probe” is a substance, e.g., a molecule, which can be specifically recognized by a particular target. Commonly employed labels include, but are not limited to, biotin, fluorescent molecules, radioactive molecules, chromogenic substrates, chemi-luminescence, and the like. Detection methods are well known for fluorescent, radioactive, chemiluminescent, chromogenic labels, as well as other commonly used labels. In the preferred embodiment of the invention, the probe is SEQ ID No. 7.

The preferred method of verifying the amplification product of nested PCR, further disclosed in the example, is by biotinylating one of the primers of inner primer pairs. The biotinylated product of the second amplification stage could be identified using luminometer. Other methods of verifying the amplification product may be used in combinatory to increase the accuracy of detecting BEFV in suspected samples.

Kits of Invention

The present invention also provides a kit for detecting the presence or absence of bovine ephemeral fever virus (BEFV) in a sample using a nested polymerase chain reaction, comprising
(i) an outer pair of oligonucleotide primers selected from the group consisting of
(a) SEQ ID NOS: 1 and 2,
(b) SEQ ID NOS: 1 and 3,
(c) SEQ ID NOS: 4 and 2,
(d) SEQ ID NOS: 4 and 3, and
(ii) an inner pair of oligonucleotide primers SEQ ID NOS: 5 and 6.

The kit of claim 1, wherein the nested PCR reaction is performed in one tube.

In the preferred kit, the outer pair of oligonucleotide primers is SEQ ID NOS: 1 and 2.

In the more preferred embodiments, the present kit can include the probe SEQ ID NO. 7, streptavidin horse raddish peroxidase (SA-HRP) and its substrates, 5× standard saline citrate (SSC) buffer and/or 05% sodium dodecyl sulphate (SDS).

In the most preferred embodiment, the present kit includes the probe coupled to magnetic beads to expedite PCR reaction.

The probe can be specifically recognized by a particular target. Commonly employed labels include, but are not limited to, biotin, fluorescent molecules, radioactive molecules, chromogenic substrates, chemi-luminescence, and the like. Detection methods are well known for fluorescent, radioactive, chemiluminescent, chromogenic labels, as well as other commonly used labels.

The following experiments are offered by way of illustration and not by way of limitation.

### EXAMPLES

The examples below are non-limiting and are merely representative of various aspects and features of the present invention.

Materials and Methods

### Example 1

### Virus Strains and Virus Propagation

Five BEFV strains, including 9316, 90126, 9333, 9310, and 9309 strains were isolated from Taiwan from different farms during 2001 to 2004 and stored in the Tainan Hsien Livestock Disease Control Center. These BEFV strains were propagated in baby hamster kidney (BHK-21) cells. These viruses were purified by plaque purification three times in BHK-21 cells and further purified by sucrose-gradient centrifugation. The identity of the virus was confirmed by reverse-transcription and polymerase chain reaction (RT-PCR), indirect-fluorescent assay, and negative stain using transmission electron microscope. The pathogenicity of the virus was confirmed by animal-inoculation experiment. These viruses were propagated in BHK-21 cells at 37° C. in Dulbecco's modified Eagle's medium supplemented with 100 μg/ml kanamycin, 2.5 μg/ml fungizone, and 10% fetal bovine serum.

### Example 2

### RNA Preparation

For comparison of RNA extraction methods, there were two commercial kits used for extraction of BEFV RNA, including RNA isolation kit (Qiagen Co., Valencia, Calif., USA) and RNA Solv Reagent (Omega Co., Doraville, Ga., USA). RNA was extracted from BEFV-infected BHK-21 cells according to manufacturer's protocol. A series of 10-fold dilutions were made, by diluting the RNA with diethylpyrocarbonate (DEPC)-treated water and tested by RT-PCR using the primer pair G2F/G2R (Table 1).

TABLE 1

Primers and probes used in real-time RT-PCR, conventional
RT-PCR, and Assay of the present invention

| | | | | |
|---|---|---|---|---|
| Label | Orientation | Assay | Sequence (5′-3′) | Position |

| | | | | |
|---|---|---|---|---|
| BEFV-F | Forward | Real-time RT-PCR | TCTAGGAGGATTCATTTGTAGGA | 219-241 |
| BEFV-R | Reverse | | GGACATAGAATTCTATTTCTTGATTCAT | 461-439 |
| BEFV-TM2 | Probe | Real-time RT-PCR | TACCCTCCTGCTGGATGCTTTTGGAG | 385-409 |
| G2F | Forward | First round PCR^{a} | GACATGGATACCAGAAGTGAGAGTC | 567-591 |
| G2R2 | Reverse | | GGAGAACATCAACAAGAG | 1106-1123 |
| G2F2 | Forward | First round PCR^{a} | CTCCTTGTACTATTAATGAC | 509-528 |
| G2R1 | Reverse | | GTAGAGTTCAAAGGATTG* | 1076-1094 |
| G2F2 | Forward | First round PCR^{a} | CTCCTTGTACTATTAATGAC | 509-528 |
| G2R2 | Reverse | | GGAGAACATCAACAAGAG | 1106-1123 |
| G2F^{b} | Forward | RAPID BAP-BEF^{a} | GACATGGATACCAGAAGTGAGAGTC | 567-591 |
| G2R1^{b} | Reverse | | GTAGAGTTCAAAGGATTG* | 1076-1094 |
| P2-2 | Forward | RAPID BAP-BEF^{c} | GGCAGAATTAGAACATGAACG | 900-920 |
| G2R^{d} | Reverse | | GAGCAAACAAGTTGGCAAG | 1027-1045 |
| P2-1^{e} | Probe | RAPID BAP-BEF | CCACAAGACCAGGAAGAGATTA | 992-1013 |
| G2F | Forward | RNA extraction^{f} | GACATGGATACCAGAAGTGAGAGTC | 567-591 |
| G2R | Reverse | | GAGCAAACAAGTTGGCAAG | 1027-1045 |

^{a}Primers tested in the first round PCR;
^{b}Primers used in conventional RT-PCR;
^{c}Primers used in the second round PCR;
^{d}Primer labeled with biotin;
^{e}Probe modified with 5′- terminal amino group;
^{f}Primers used to test the efficiency of RNA extraction.
*The primers deleted one nucleotide from the sequence of original position.

### Example 3

### Primers and Probes

The primers and probes for the assay of the present invention, TaqMan RT-PCR, and conventional RT-PCR was designed by comparing multiple sequence of BEFV G gene using DNASTAR software package (DNASTAR Inc., Madison, Wis., USA). The highly conserved regions among BEFV isolates were selected for the design of primers and probes (Table 1). The primers and the probe were synthesized by TIB Molbiol Inc. (Berlin, Germany).

### Example 4

### Assay of Invention

The nested PCR was performed in a thermal reactor (Thermocycler, TaKaRa, Japan).

In the reverse transcription and first amplification, one-tube RT-PCR was carried out. After RNA and forward-primer were denatured at 70° C. for 5 minutes, 44 μl of One-tube RT-PCR reagent (Tris-HCl, MgCl₂, dATP, dGTP, dTTP, dCTP, external primer, Reverse transcriptase, Rnase inhibitor, and AmpliTag DNA polymerase) for first round PCR was added to the tube. 25 μl of RT-PCR product was transferred to another tube containing 25 μl of amplification reagent (Tris-HCl, MgCl₂, dATP, dGTP, dTTP, dCTP, internal primer, and AmpliTag DNA polymerase) for Nested-PCR.

The primers for nested PCR were derived from the Bovine ephemeral fever virus genome, encoding the G protein with sequence of the

external primer:
5′-GACATGGATACCAGAAGTGAGAGTC-3′
and
5′-GTAGAGTTCAAAGGATTG-3′
and
internal primers
5′-GGCAGAATTAGAACATGAACG-3′
and
5′-biotin-GAGCAAACAAGTTGGCAAG-3′.

For each assay, one negative control was prepared. The RT-PCR was carried out in a thermal reactor. After 1 hour incubation at 37° C. for reverse transcription, the following reaction was the same as Nested-PCR. cDNA was first denatured at 95° C. for 5 min, and the amplification was performed for 35 cycles of 95° C. for 30 s, 50° C. for 30 s, and 72° C. for 45 s. After the last cycle, the samples were incubated for 5 min at 72° C. The second amplification was performed with the same extension program except the external primer was replaced by the internal primer.

In a hybridization tube, 10 μl of each amplified DNA sample and 290 μl of hybridization reagent, containing 15 μl of MagProbe (beads with probe: 5′-amine-CCACAAGACCAGGAAGAGATT-3′) was added, vortexed, and incubated at 95° C. for 5 min and at 50° C. for 20 min in a dry bath. Tubes were then transferred to magnetic wells for 5 min. The hybridization buffer was removed by aspiration without disturbing the MagProbe. After adding 1 ml of preheated 50° C. wash buffer, the tubes were vortexed and held in a magnetic well for 5 min. The wash buffer was removed by aspiration. The wash steps were then repeated once again.

After adding blocking solution and streptavidin-horseradish peroxidase, the hybridization tube was vortexed, kept from light at room temperature for 20 min, and transferred back to the magnetic well for another 5 min. Then the supernatant was removed by careful aspiration. After washing twice and resuspending with phosphate-buffered saline, the tube was placed in a luminimeter to determine the number of relative light units (RLU) produced by the reaction. The RLU is directly proporational to the quantities of PCR products that hybridize with magnetic beads-labeled with a probe.

The sensitivity and specificity of the assay of the present invention were also examined. To test the sensitivity of the assay, plasmid pGEM-T-Easy with the full-length G protein-encoding gene of BEFV diluted serially from 1 to 10⁷ copies/μl was detected by the assay. A series of 10-fold dilutions of BEFV-infected BHK-21 cells were made to test the specificity of the assay. Five BEFV strains, including 9316, 90126, 9333, 9310, and 9309 strains described above were examined.

### Example 5

### Conventional RT-PCR and TaqMan RT-PCR

Conventional one-tube RT-PCR was performed with the same PCR program and a primer set (G2F/G2R1) used in the Assay of the present invention (Table 1). PCR products (528 bp) were purified individually with gel purification kit (Amersham Phamacia Biotech, USA) and sequenced using the DNA sequencing kit (PE Applied Biosystems, USA). The sequences were analyzed using the Clustal program of DNASTAR software package (DNASTAR Inc.).

In the reverse transcription, after the addition of 1 μl of 20 U RNase inhibitor and 0.5 μl of 200 U/ml reverse transcriptase (Promega, USA), samples were incubated at 42° C. for 40 min. Subsequently, the RT reaction was terminated by enzyme inactivation at 83° C. for 5 min. The TaqMan RT-PCR was performed in a LightCycler (Roche, Germany) by using LightCycler TaqMan Master kit (Roche). The primers and probe used in TaqMan PCR were shown in Table 1. PCR reactions were subjected to 45 cycles consisting of denaturation at 95° C. for 10 s, annealing at 58° C. for 20 s, and extension at 72° C. for 5 s and a final extension at 72° C. for 7 min.

### Example 6

### Optimal Conditions for Assay of Invention

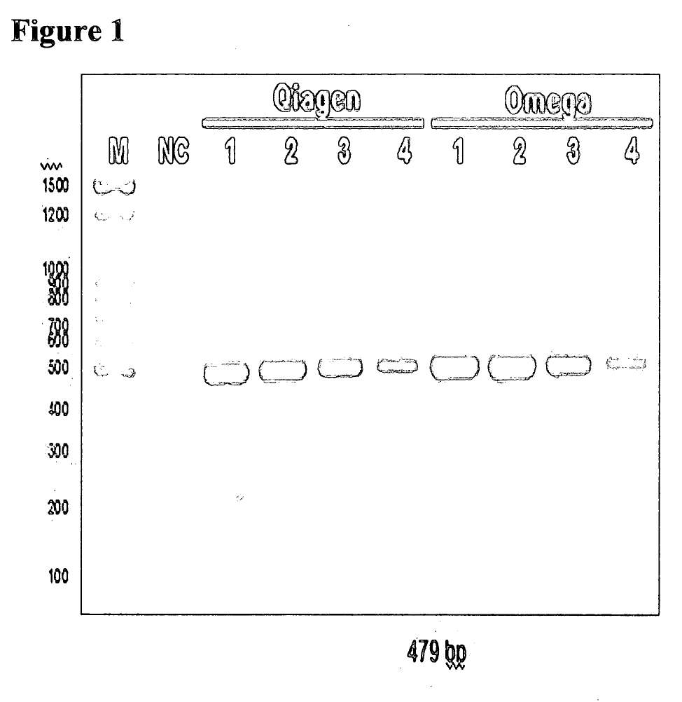

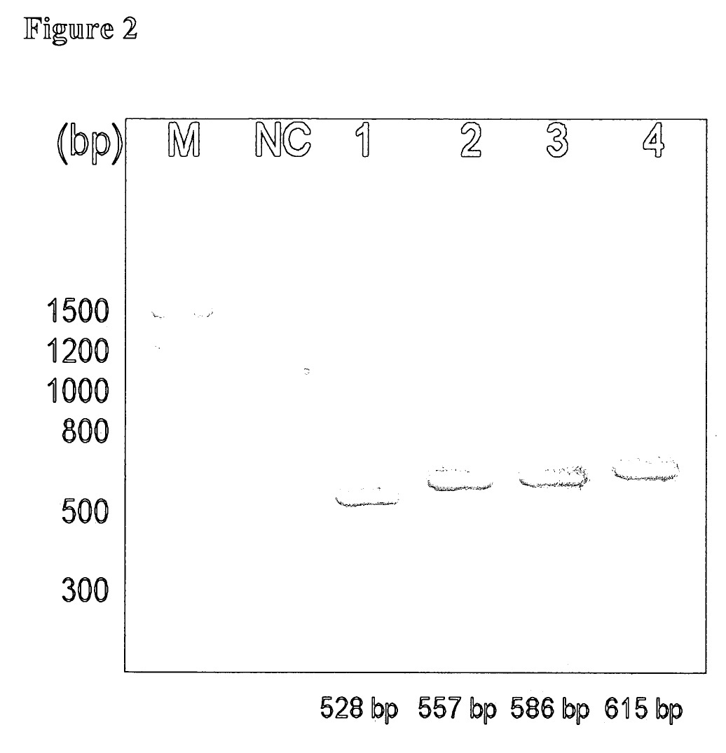

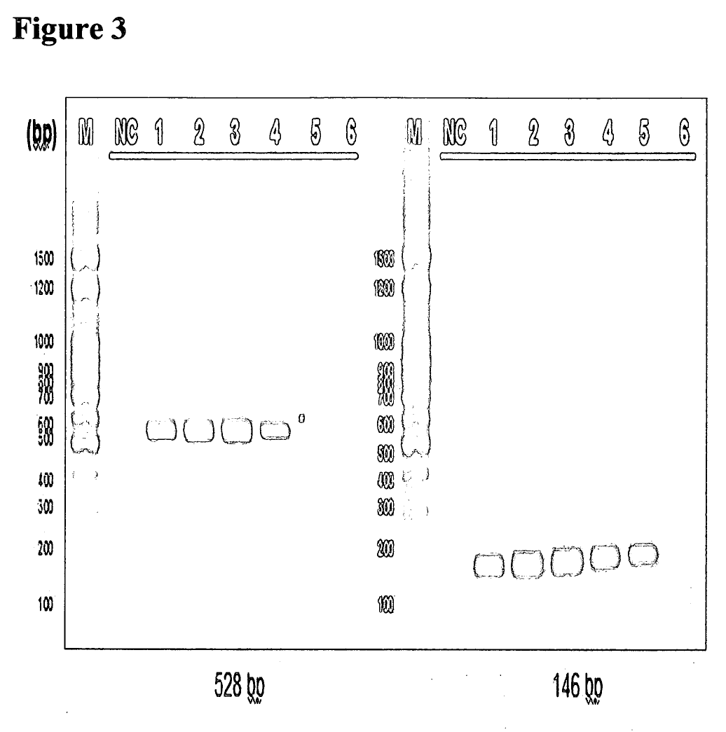

A direct sensitivity comparison of two RNA extraction methods following amplification of 10-fold diluted RNA extracts showed that these two methods could extract RNA reliably from BEFV-infected BHK-21 cells and had the same sensitivity. FIG. 1 shows that both methods gave reliable detection when using RT-PCR assay. In the present invention, RT-PCR was preliminary optimized by testing various reverse transcriptase, the primer pairs, and annealing temperatures. Examination of the amplified PCR products following electrophoresis in agarose gels showed the expected sizes of PCR products using four tested sets of primers (FIG. 2) in the first round PCR. The PCR-amplified products revealed the almost identical mobilities in 1.2% agarose gels by using the selected primer sets of G2F/G2R1 and P2-2/G2R at different annealing temperatures (FIG. 3). No amplification occurred in the first PCR reactions at 60° C. but positive by nested PCR using the same annealing temperature (FIG. 3). This suggested that nested PCR showed more sensitive than single-round PCR as reported previously (Liu, H. J. et al., 1999, Identification of the σ C-encoded gene of avian reovirus by nested PCR and restriction endonuclease analysis. J. Virol. Methods. 81: 83-90; Wang, J. Y. et al., 2004, Performance assessment of a nested PCR assay (the RAPID BAP-MTB) and the BD ProbeTec ET system for detection of Mycobacterium tuberculosis in clinical specimens. J. Clin. Microbiol. 42: 4599-4603). The PCR products were also sequenced to confirm the specificity of PCR assay. Based on our results, the nested PCR procedure was optimized by using the AMV reverse transcriptase for reverse transcription, the primer sets of G2F/G2R1 and P2-2/G2R, and annealing temperature at 50° C.

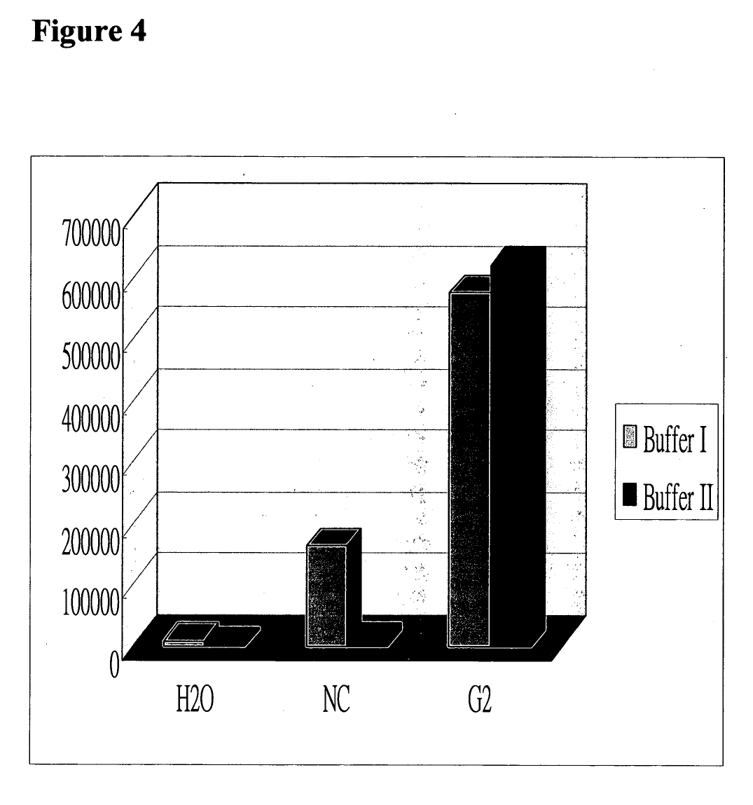

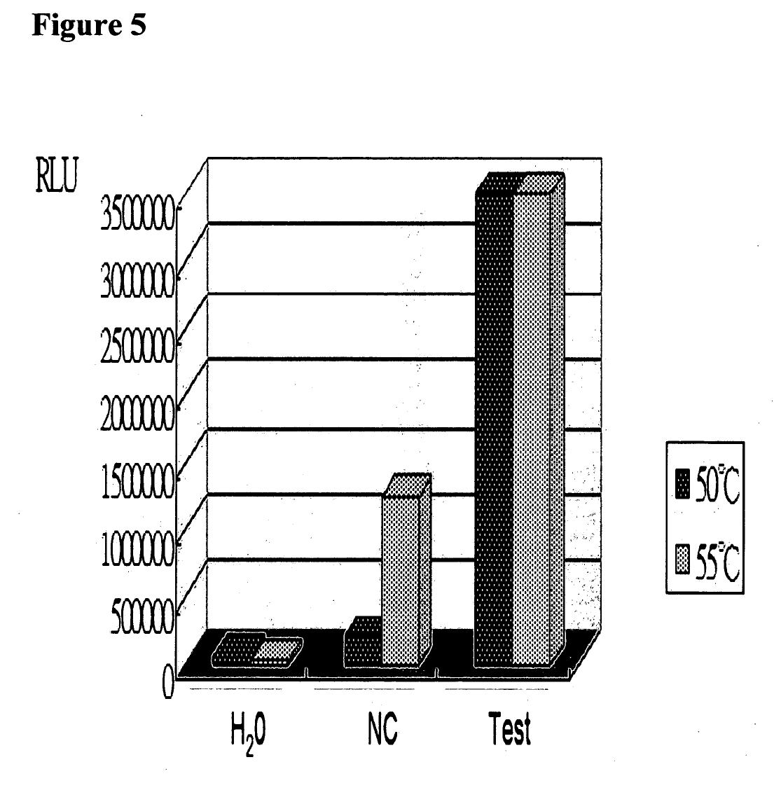

Different hybridization temperatures and reagents were also tested for the assay of the present invention. The lower background obtained from negative controls were determined to be the use of buffer II for hybridization at 50° C. (FIGS. 4 and 5). Thus, the optimal hybridization conditions at 50° C. in 5×SSC and 0.5% SDS showed clear discrimination between negative control and tested samples (FIGS. 4 and 5). The hybridization was complete within 20 min.

### Example 7

### Sensitivity and Specificity of Assay of Invention

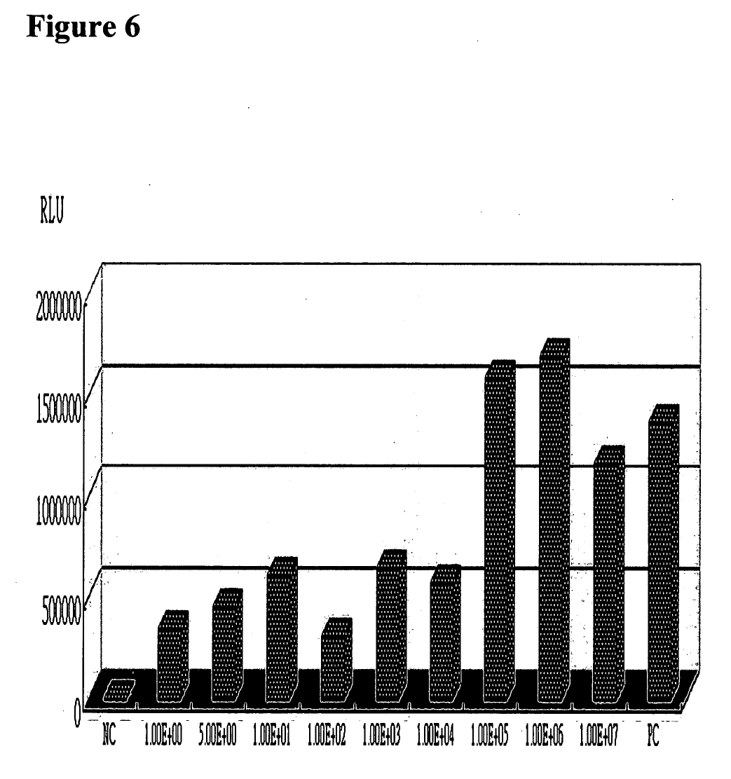

To determine the end point of sensitivity, a series of 10-fold dilutions of BEFV plasmid DNA were mad and analyzed by the assay. As shown in FIG. 6, there is a clear distinction between negative control and all tested samples. The assay is highly sensitive as it can detect samples ranging from 1 to 10⁷ copies/μl as shown in FIG. 6. The detection limit of the assay was 1 copy/μl (FIG. 6).

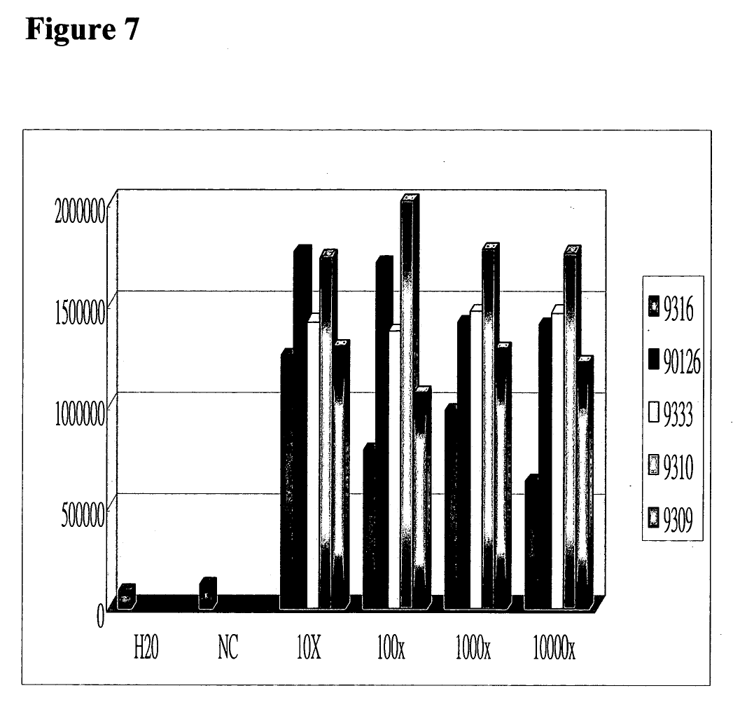

Specific detection was also demonstrated for BEFV isolates by the assay. The assay exhibited highly specific as all negative controls failed to show any positive detection. The sample-to-negative control ratio was greater than 300 in a 10,000-fold dilution of five BEFV isolates (FIG. 7).

Testing the present invention, all positive signals were significantly stronger than the backgrounds. Some condition could attain 27 times stronger than the backgrounds. Given the above, the positive signals were sufficient to differentiate presence or absence of BEFV.

### Example 8

### Comparison of Diagnostic Value of Assay of Invention

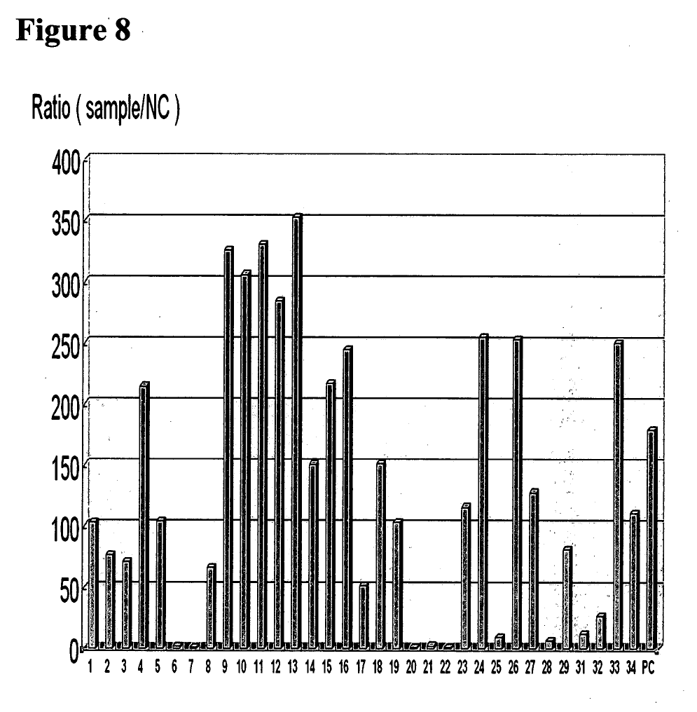

To increase the sensitivity and specificity of the assay of the present invention, the nested PCR in combination with magnetic beads-based DNA probing technology was performed to amplify the G protein-encoding gene of BEFV. In the 34 blood specimens, there were 25 samples exhibited RAPID BAP-BEF positive and the ratio was greater than 20 in these samples (FIG. 8). There were 19 blood samples that tested negative by conventional RT-PCR with primer pair G2F/G2R1 but positive by the assay of the present invention with the same set of primers (Table 2).

TABLE 2
Comparison of the diagnostic sensitivity of real-time RT-PCR,
conventional RT-PCR, and assay of the present invention in the
detection of 34 BEF-suspected clinical samples.

| Assay of | | | |
|---|---|---|---|
| the present invention | | Real time RT-PCR | Conventional RT-PCR |
| Positive | 25 | 12 | 6 |
| Negative | 9 | 24 | 28 |
| Sensitivity (%) | 72.73% | 36.36% | 18.18% |

Table 2 showed comparisons of the results of BEFV detection with the three assays in blood samples. In the 34 blood samples collected from different farms during 2001 to 2004 in Taiwan, the results were the assay of the invention and conventional RT-PCR and real-time RT-PCR negative in 19 and 13 samples, respectively. The diagnostic sensitivity of the assay of the present invention, real-time RT-PCR, and conventional RT-PCR in the detection of 34 clinical blood samples suspected to have BEFV infections was 72.73%, 36.36%, and 18.18%, respectively (Table 2). The results indicated that the novel assay of the present invention showed more sensitive than the conventional RT-PCR and real-time RT-PCR assays for the detection of BEFV.

Table 3 futher stated the result of the detection stated in Table 2. The positives indicated by “x” were identified according to the sample's RLU ratio (Sample/NC). The positives identified by the present invention were #1, #2, #3, #4, #5, #8, #9, #10, #11, #12, #13, #14, #15, #16, #17, #18, #19, #23, #24, #26, #27, #29, #33 and #34 (Table 3).

TABLE 3
The result of the virus detection

| | The present | | Real-time |
|---|---|---|---|
| Sample # | invention | RT-PCR | PCR |
| 1 | x | | |
| 2 | x | x | x |
| 3 | x | | x |
| 4 | x | | |
| 5 | x | x | x |
| 6 | | | |
| 7 | | | |
| 8 | x | x | x |
| 9 | x | | |
| 10 | x | | x |
| 11 | x | | |
| 12 | x | | |
| 13 | x | | |
| 14 | x | | |
| 15 | x | x | |
| 16 | x | | x |
| 17 | x | x | x |
| 18 | x | | |
| 19 | x | | |
| 20 | | | |
| 21 | | | |
| 22 | | | |
| 23 | x | x | x |
| 24 | x | | x |
| 25 | | | |
| 26 | x | | x |
| 27 | x | | |
| 28 | | | |
| 29 | x | | |
| 31 | | | |
| 32 | x | | |
| 33 | x | | x |
| 34 | x | | x |

The novel assay of the present invention was highly specific as all negative controls failed to show any positive detection. All positive detection which had the sample-to-negative control ratios of greater than 20 by the assay showed clear discrimination between negative control and tested samples. The results suggested that using a cutoff value of greater than twenty times the negative control for positive was appropriate. It also showed a high level of sensitivity as the detection limit of the assay was assessed at 1 copy/μl. In addition, the diagnostic sensitivity of the nested PCR-based assay of the present invention is superior to that of the conventional RT-PCR and real-time RT-PCR for diagnosis of clinical blood samples of BEF. Due to its higher sensitivity and specificity, the assay is well suited for detection of the early stage of BEFV infections. In conclusion, the novel assay of the present invention is a sensitive, rapid, and reliable method to ascertain BEFV in cultured cells or blood specimens.

One skilled in the art readily appreciates that the present invention is well adapted to carry out the objects and obtain the ends and advantages mentioned, as well as those inherent therein. The cell lines, embryos, animals, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

Sequence List

<100>GENERAL INFORMATION
<160>NUMBER OF SEQ ID NOS: 8
<200>SEQUENCE CHARACTERISTICS:
<210>SEQUENCE ID NO. 1
<211>Length: 25
<212>Type: DNA
<213>Organism: Artificial Sequence
<223>OTHER INFORMATION: forward primer

<400>Sequence: 1

5′-GACATGGATACCAGAAGTGAGAGTC-3′

200>SEQUENCE CHARACTERISTICS:
<210>SEQUENCE ID NO. 2
<211>Length: 18
<212>Type: DNA
<213>Organism: Artificial Sequence
<223>OTHER INFORMATION: reverse primer

<400>Sequence: 2

5′-GTAGAGTTCAAAGGATTG-3′

<200>SEQUENCE CHARACTERISTICS:
<210>SEQUENCE ID NO 3
<211>Length: 18
<212>Type: DNA
<213>Organism: Artificial sequence
<223>OTHER INFORMATION: reverse primer

<400>Sequence: 3

5′-GGAGAACATCAACAAGAG-3′

<200>SEQUENCE CHARACTERISTICS:
<210>SEQUENCE ID NO. 4
<211>Length: 20
<212>Type: DNA
<213>Organism: Artificial Sequence
<223>OTHER INFORMATION: forward primer

<400>Sequence: 4

5′-CTCCTTGTACTATTAATGAC-3′

200>SEQUENCE CHARACTERISTICS:
<210>SEQUENCE ID NO. 5
<211>Length: 21
<212>Type: DNA
<213>Organism: Artificial Sequence
<223>OTHER INFORMATION: forward primer

<400>Sequence: 5

5′-GGCAGAATTAGAACATGAACG-3′

<200>SEQUENCE CHARACTERISTICS:
<210>SEQUENCE ID NO 6
<211>Length: 19
<212>Type: DNA
<213>Organism: Artificial Sequence
<223>OTHER INFORMATION: reverse primer

<400>Sequence: 6

5′-GAGCAAACAAGTTGGCAAG-3′

<200>SEQUENCE CHARACTERISTICS:
<210>SEQUENCE ID NO 7
<211>Length: 22
<212>Type: DNA
<213>Organism: Artificial Sequence
<223>OTHER INFORMATION: probe specific to bovine ephemeral fever virus

<400>Sequence: 7

5′-CCACAAGACCAGGAAGAGATTA-3′

What is claimed is:

1. A kit for detecting the presence or absence of bovine ephemeral fever virus (BEFV) in a sample using a nested polymerase chain reaction, comprising
(i) an outer pair of oligonucleotide primers selected from the group consisting of
(a) SEQ ID NOS: 1 and 2,
(b) SEQ ID NOS: 1 and 3,
(c) SEQ ID NOS: 4 and 2,
(d) SEQ ID NOS: 4 and 3, and
(ii) an inner pair of oligonucleotide primers SEQ ID NOS: 5 and 6.

2. The kit of claim 1, wherein the nested PCR reaction is performed in one tube.

3. The kit of claim 1, wherein the outer pair of oligonucleotide primers is SEQ ID NOS: 1 and 2.

4. The kit of claim 1, wherein the inner pair of oligonucleotide primers is labeled by a detectable group selected from the group consisting of fluorescent molecules, radioactive molecules, chromogenic substrates, biotin, acridinium ester and acridinium-9-carboxamide.

5. The kit of claim 4, wherein the detectable group is biotin.

6. The kit of claim 1, further comprises a probe wherein the sequence of the probe is SEQ ID NO. 7.

7. The kit of claim 6, wherein the probe is coupled to magnetic beads.

8. The kit of claim 1, further comprises streptavidin horse raddish peroxidase (SA-HRP) and its substrates.

9. The kit of claim 1, further comprises 5× standard saline citrate (SSC) buffer.

10. The kit of claim 1, further comprises 05% sodium dodecyl sulphate (SDS).

11. A method for detecting the presence or absence of BEFV in a sample using a nested polymerase chain reaction, comprising
(vi) adding into one tube with RNA from the sample, reverse transcriptase, buffer, dNTP, Taq polymerase, an outer pair of oligonucleotide primers selected from the group consisting of
(a) SEQ ID NOS: 1 and 2,
(b) SEQ ID NOS: 1 and 3,
(c) SEQ ID NOS: 4 and 2, and
(d) SEQ ID NOS: 4 and 3,
(vii) performing the first-stage polymerase chain reaction of the PCR products from the outer primers;
(viii) adding into one tube with template from (ii), buffer, dNTP, Taq polymerase, and an inner pair of oligonucleotide primers SEQ ID NOS: 5 and 6;
(ix) performing the second-stage polymerase chain reaction of the PCR products from for the inner primers; and
(x) identifying BEFV by the probe having SEQ ID NO: 7.

12. The method of claim 11, wherein the outer pair of oligonucleotide primers is SEQ ID NOS: 1 and 2.

13. The method of claim 11, wherein the inner pair of oligonucleotide primers is labeled by a detectable group selected from the group consisting of fluorescent molecules, radioactive molecules, chromogenic substrates, biotin, acridinium ester and acridinium-9-carboxamide.

14. The method of claim 13, wherein the detectable group is biotin.

15. The method of claim 11, wherein the probe is coupled to magnetic beads.

16. The method of claim 11, wherein the identification is determined by hybridization reaction carried out at 30-60° C., under 1-10× standard saline citrate (SSC) buffer and 0.1-2% sodium dodecyl sulphate (SDS).

17. The method of claim 16, wherein hybridization reaction is carried out at 50° C., under 5× standard saline citrate (SSC) buffer and 0.5% sodium dodecyl sulphate (SDS).

18. The method of claim 11, which could be applied to detect the presence of one copy of BEFV in a sample.

19. The method of claim 18, which could be applied to detect the early stage of BEFV infections in a sample.

20. A nucleotide sequence for detecting the presence or absence of bovine ephemeral fever virus (BEFV) is selected from the group consisting of

| | |
|---|---|
| GACATGGATACCAGAAGTGAGAGTC | (SEQ ID NO: 1) |
| GTAGAGTTCAAAGGATTG | (SEQ ID NO: 2) |
| GGAGAACATCAACAAGAG | (SEQ ID NO: 3) |
| CTCCTTGTACTATTAATGAC | (SEQ ID NO: 4) |
| GGCAGAATTAGAACATGAACG | (SEQ ID NO: 5) |
| GAGCAAACAAGTTGGCAAG | (SEQ ID NO: 6) |
| CCACAAGACCAGGAAGAGATTA. | (SEQ ID NO: 7) |


SEQUENCE LISTING (11 sequences)

SEQ ID NO: 1 - DNA, 25 bp, Bovine ephemeral fever virus
gacatggataccagaagtgagagtc

SEQ ID NO: 2 - DNA, 18 bp, Bovine ephemeral fever virus
gtagagttcaaaggattg

SEQ ID NO: 3 - DNA, 18 bp, Bovine ephemeral fever virus
ggagaacatcaacaagag

SEQ ID NO: 4 - DNA, 20 bp, Bovine ephemeral fever virus
ctccttgtactattaatgac

SEQ ID NO: 5 - DNA, 21 bp, Bovine ephemeral fever virus
ggcagaattagaacatgaacg

SEQ ID NO: 6 - DNA, 19 bp, Bovine ephemeral fever virus
gagcaaacaagttggcaag

SEQ ID NO: 7 - DNA, 22 bp, Bovine ephemeral fever virus
ccacaagaccaggaagagatta

SEQ ID NO: 8 - DNA, 23 bp, Bovine ephemeral fever virus
tctaggaggattcatttgtagga

SEQ ID NO: 9 - DNA, 28 bp, Bovine ephemeral fever virus
ggacatagaattctatttcttgattcat

SEQ ID NO: 10 - DNA, 26 bp, Bovine ephemeral fever virus
taccctcctgctggatgcttttggag

SEQ ID NO: 11 - DNA, 21 bp, Bovine ephemeral fever virus
ccacaagaccaggaagagatt